(19) United States
(12) Patent Application Publication
(10) Pub. No.: US 2003/0164169 A1
(43) Pub. Date: Sep. 4, 2003

(54) Inhalation device and method for production of a particulate mist for inhalation purposes

(76) Inventors: Roland Stangl, Moosburg (DE); Manfred Keller, Bad Grotzingen (DE)

Correspondence Address: MERCHANT & GOULD PC, P.O. BOX 2903, MINNEAPOLIS, MN, 55402-0903, US

(21) Appl. No.: 10/344,916

(22) PCT Filed: Aug. 20, 2001
(86) PCT No.: PCT/EP01/09589

(30) Foreign Application Priority Data
Aug. 18, 2000 (DE) 100 40 528.2

(51) Int. Cl. A61M 16/00

(52) U.S. Cl. 128/203.12; 128/203.15

(57) ABSTRACT

The inhalation device comprises an oscillator (1), on which a powder (6) for nebulisation may be placed, containing a medicament and which may be activated in order to nebulise the applied powder, a mixing chamber (2) into which the powder particles are thrown and where an aerosol cloud (6a) is formed from incoherent particles, an inhalation check valve (3) which permits ambient air into the mixing chamber (2) during inhalation to evacuate the particle cloud (6a) and an exhalation check valve (4), by means of which expired air is led off into the environment to prevent damp breath entering the mixing chamber (2) during exhalation.

The invention relates to an inhalation device having an oscillator, preferably an oscillating membrane, for nebulising medicaments in a powdered form. The invention furthermore relates to a method for producing a particulate mist for inhalation purposes.

An inhalation device of the type in question here is known, for example, from U.S. 5,694,920. In order to nebulise a medicament in powdered form for inhalation by a patient, the medicament in powdered form is transported in the known inhalation device out of a container by means of a piezoelectric oscillator, is thereby deagglomerated and converted into a fluid-like state. The oscillator comprises an oscillating membrane from which the oscillations are transferred to the medicament in powdered form in the container. Particles having a size in the range of 1 to 5 μm are produced in a comparatively small space which is separate from the air stream. Only by effect of an electrostatic field do the particles in the desired size range arrive in the air stream of the inhaling patient whereas larger particles remain in the space or container. A sensor detects the breath flow rate in the inhalation device and emits an output signal to a control means which activates the oscillator when a predetermined minimum value is reached and thus triggers nebulisation of the powdered medicament. Generation of the electrostatic field occurs at the same time, which is indispensable in the known device for the provision of particles in the desired size range.

Described in WO 97/26934, which shows an inhalation device constructed in a very similar manner, is a controller which controls the energy supplied to the oscillator to nebulise the powder.

Against this background, the object forming the basis for the invention is thus to provide an improved inhalation device having an oscillator to nebulise medicaments in powdered form, which is more simply constructed and easier to control in comparison to the known devices of this type, without having to accept a reduction in dosage accuracy and manageability.

This object is solved by an inhalation device having an oscillator on which a powder for nebulisation which contains a medicament may be placed and which may be activated in order to nebulise the applied powder, a mixing chamber into which the powder particles arc thrown and where an aerosol cloud is formed from incoherent particles, an inhalation check valve which permits ambient air into the mixing chamber during inhalation to evacuate the particle cloud, and an exhalation check valve, by means of which expired air is led off into the environment to prevent damp breath from entering the mixing chamber during exhalation.

A method for the production of a particulate mist containing a medicament for inhalation by a patient during at least one respiratory cycle can be carried out using the inhalation device according to the invention, said method having the following steps:

detection of the end of the exhalation phase of the respiratory cycle;

placing of a predetermined amount of a powder containing a medicament onto the membrane of an oscillator;

activation of the oscillator to deagglomerate the amount of powder during a first stage of the inhalation phase of the respiratory cycle; and

deactivation of the oscillator following completion of the deagglomeration of the amount of powder.

In an advantageous development of the invention, the method includes the detection and evaluation of the amount of respiratory cycles and different inspiratory parameters as well as the display of the results.

The inhalation device according to the invention provides the energy required to deagglomerate the powder containing a medicament by oscillating a membrane, the amplitude and oscillation energy of which can be regulated and controlled. The energy is transferred to the powder by means of a rapidly vibrating element (e.g. a piezooscillator). The bonds of the powder particles are loosened or broken by means of vibration effects. The powder is thrown into the deagglomeration chamber where a further deagglomeration into particles which can be inhaled occurs owing to collisions of particles with other particles and with the walls. These particles form an aerosol cloud in the chamber. The patient can slowly inhale the active substance via the inhalation valve without any particular effort by means of a normal inhalation process (inspiratory flow rate of 5 to 20 litres per minute). Slow inhalation is advantageous because undesirable impaction effects in the mouth and throat area can thereby be significantly reduced since particles can follow the air stream better at lower speeds. The exhalation valve prevents moist air from entering the inhalation chamber during the desired rapid exhalation. A rapid exhalation is advisable since the particles can thereby impact and become lodged in the lower bronchial tract, thus preventing the particles from being exhaled again.

A particularly advantageous embodiment of the inhalation device according to the invention includes a sensor means for detecting inhalation by the patient. When the patient begins to breathe, the oscillator is activated via the sensor so that an aerosol cloud which is virtually triggered by the inhalation flow is produced.

The DPI according to the invention thus allows the patient to carry out an optimal inhalation process for the application and deposition of the active substance. Since the oscillation energy and thus aerosol production is controlled and triggered via a sensor system, the patient can inhale the active substance with the greatest possible efficiency in a relaxed manner and without any coordination problems.

The inhalation device according to the invention allows the application of a medicament by inhalation under optimal inhalation conditions so that a reproducible topical and systemic medicinal therapy is possible.

The inhalation device according to the invention is particularly suitable for inhalation therapies in which the application of the medicament occurs in several respiratory cycles, i.e. in smaller amounts. However, the inhalation device according to the invention can also be used for the administration of a single dose of medicament during an inhalation process.

The invention is explained in more detail below by means of an embodiment example with reference to the drawings.

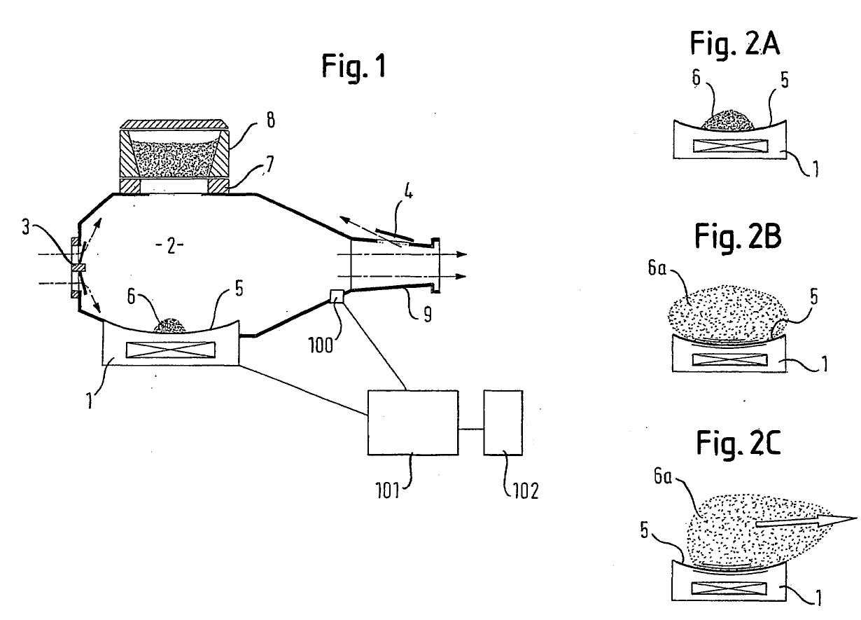

FIG. 1 shows a schematic view of an embodiment of an inhalation device according to the invention;

FIGS. 2A to 2C schematically show the progress of the nebulisation and inhalation of an amount of powder in the inhalation device according to FIG. 1;

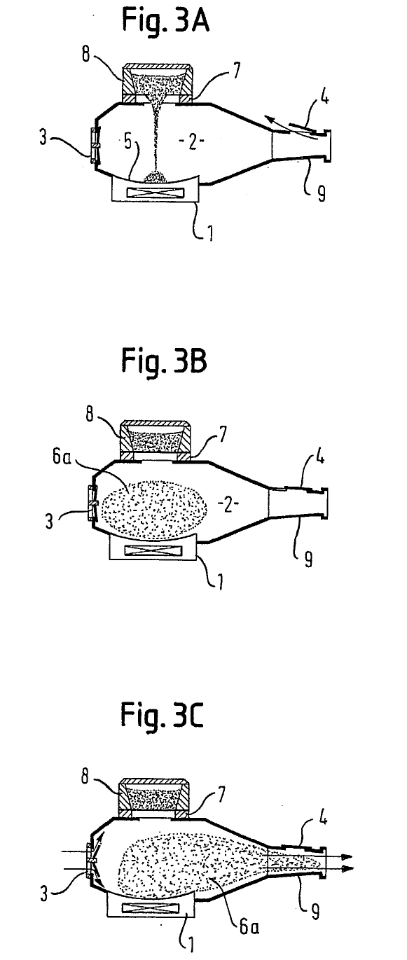

FIGS. 3A to 3C show the progress of a respiratory cycle in an inhalation device according to the invention;

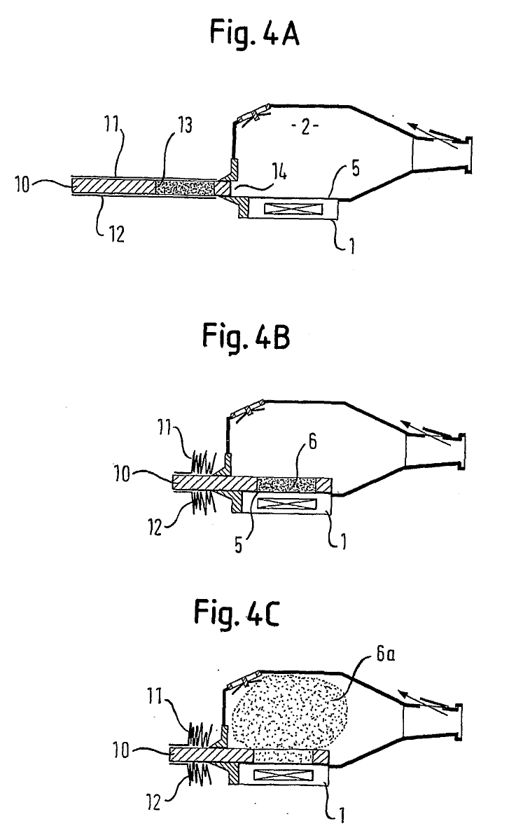

FIGS. 4A to 4C show the progress of the placing and nebulisation of an amount of powder according to a first alternative;

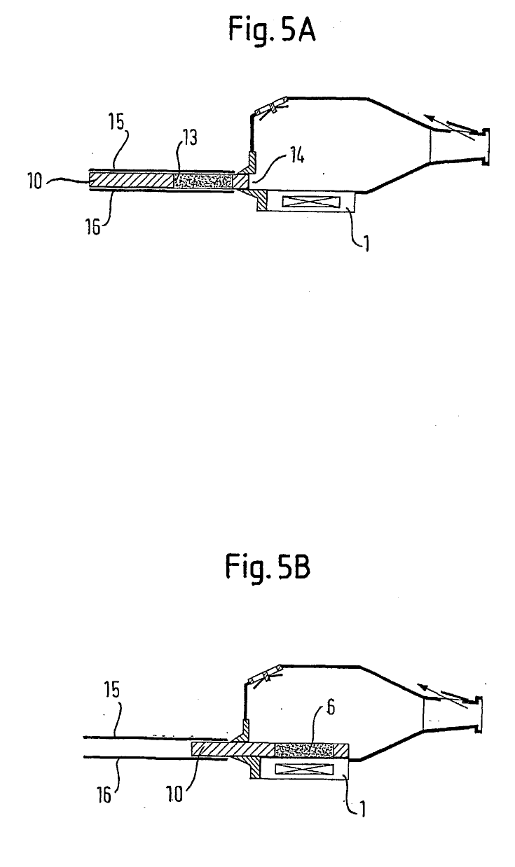

FIGS. 5A to 5B show the progress of the placing and nebulisation of an amount of powder according to a second alternative; and

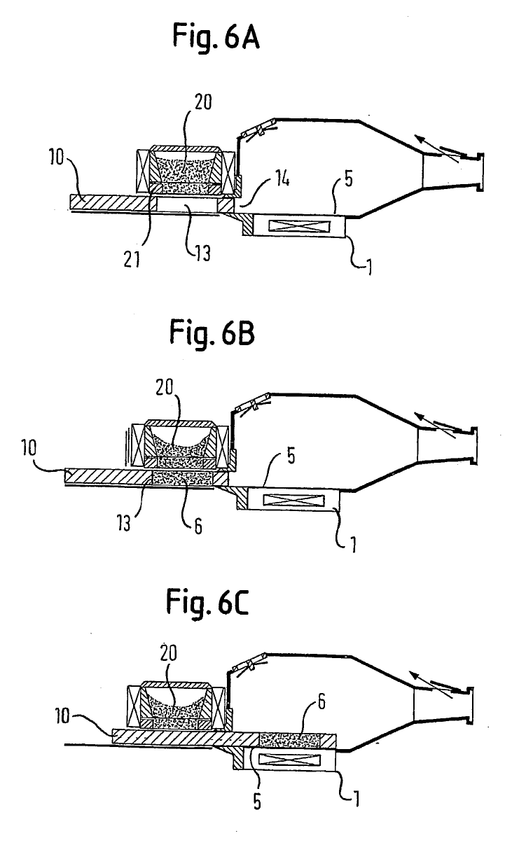

FIGS. 6A to 6C show the progress of the placing and nebulisation of an amount of powder according to a second alternative.

FIG. 1 shows an inhalation device according to the invention having an oscillator 1, a mixing chamber 2, an inhalation valve 3 and an exhalation valve 4. In FIG. 1, a medicament 6 in the form of a powder is shown on a membrane 5 of the oscillator 1, said medicament being placed on the membrane 5 of the oscillator 1 from a storage container 8 by a dosing means 7.

This state is also represented in FIG. 2A, however only the oscillator 1 with the membrane 5 and the medicament 6 in the form of a powder are shown. If the oscillator 1 is activated, the membrane 5 starts to oscillate. The oscillation of the membrane 5 is transferred, as shown in FIG. 2B, to the medicament in the form of a powder which is at least partially deagglomerated and disperses as a particle cloud 6a above the membrane 5 in the mixing chamber 2. The particle bolus is available therein to the patient for inhalation.

If the patient inhales by means of a mouthpiece 9 via the mixing chamber 2, ambient air, as shown in FIG. 1, flows into the mixing chamber 2 through the inhalation valve 3 and takes the particle cloud 6a with it, as shown in FIG. 2C, so that the patient inhales the deagglomerated medicament powder. During exhalation, the inhalation valve 3 closes so that the exhaled air of the patient practically no longer enters the mixing chamber 2 but rather flows out into the environment via the exhalation valve 4.

Both the inhalation valve 3 and the exhalation valve 4 are check valves, each having a flat, elastic valve element. In the inhalation valve 3, the flat, elastic valve element is arranged and fixed in such a way that it lifts up from the valve seat during inhalation and allows ambient air to flow into the mixing chamber 2. This leads to the decisive advantage of the inhalation device according to the invention, which consists in that the particle bolus stored in the mixing chamber 2 from the point of production is directly available to the patient during inhalation and is evacuated by means of the airflow conducted through the mixing chamber 2.

During exhalation, the flat, elastic valve element of the inhalation valve 3 lies on the valve seat and closes the mixing chamber 2. Owing to the pressure increase in the mixing chamber 2 linked with exhalation, the flat, elastic valve element of the exhalation valve 4 lifts up from the valve seat so that the exhaled air of the patient can flow out into the environment through the opened exhalation valve 4. During inhalation, the flat, elastic valve element of the exhalation valve 4 lies on the valve seat thereby closing the exhalation valve 4.

A sensor 100 is furthermore shown in FIG. 1, which detects the respiratory flow rate and emits a signal to a control means 101. The control means 101 activates the oscillator 1 if a respiratory flow rate is detected by means of the sensor 100, which indicates the end of exhalation and the beginning of inhalation. The control means 101 can be used to detect and evaluate the number of respiratory cycles and different inspiratory parameters. The results are displayed to the patient by a display means 102 in order to control the therapy session in this manner. The control means 101 can furthermore be designed, preferably programmed, in such a way that the number of breaths and the dosage released can be registered by means thereof. Both the values and the parameters derived therefrom, such as, for example, the applied dosage or the remaining content in a reservoir accommodating several doses, can then be displayed by the display means 102.

FIGS. 3A to 3C show an inhalation cycle which occurs in the inhalation device according to the invention. As is shown in FIG. 3A, during exhalation by the patient, a predetermined amount of the medicament in the form of a powder is placed on the membrane 5 of the oscillator 1 from the reservoir 8 by the dosing means 7. During this period, the exhaled air, as already described above, flows out through the exhalation valve 4, without affecting the placement of the amount of medicament. At the end of the exhalation process, as shown in FIG. 3B, the oscillator 1 is activated so that the particle cloud 6a forms in the mixing chamber 2 above the membrane 5. During inhalation, as shown in FIG. 3C, the ambient air flows through the now open inhalation valve 3 into and through the mixing chamber 2, with the air taking the particle cloud 6a with it and thus evacuating the mixing chamber 2. The patient inhales the particle cloud together with the inflow of ambient air. At the end of inhalation, the mixing chamber 2 is evacuated and the patient exhales into the inhalation device according to the invention, and thus the state according to FIG. 3A is achieved again.

As an alternative to the introduction of the powder containing a medicament as described hitherto, the powder can also be introduced as shown in FIGS. 4A to 4C, 5A to 5B or 6A to 6C.

Shown in FIG. 4A is an embodiment in which introduction occurs by means of a reservoir device 10 having cavities for storing a powder. The reservoir device 10 comprises a water vapour-tight and airtight film 11 and 12 on the upper and lower sides. A predetermined amount of powder 13 is stored in the cavities of the reservoir device. The carrier plate 10 is inserted into the mixing chamber 2 by means of an opening 14 in such a way that the sealing films 11 and 12 arranged on the upper and lower sides are held back or removed, with the medicament 6 in powder form thereby directly coming to rest on the membrane 5 of the oscillator 1, as shown in FIG. 4B. It is nebulised from there when the oscillator 1 is activated, as is shown in FIG. 4C.

A reservoir device 10 is also used in the embodiment according to FIG. 5A, in which the powder 6 is stored in the cavities 13. Instead of films, rigid covers 15 and 16 are arranged on the upper and lower sides, which are pushed back during insertion of the reservoir device 10 into the opening 14 of the mixing chamber provided therefor, as is shown in FIG. 5B.

In the embodiment according to FIG. 6A, a powder reservoir 20 is provided with a dosing device 21 which doses the amount of powder to be nebulised into the cavity 13 of a reservoir device 10. In FIG. 6A, the state before filling of the dosing container 13 is shown. In FIG. 6B, a predetermined amount of powder 6 is fed into the dosing container 13. In FIG. 6C, the filled dosing container 13 is located above the membrane 5 of the oscillator 1.

1. An inhalation device having
an oscillator (1) on which a powder (6) for nebulisation containing a medicament may be placed and which may be activated in order to nebulise the applied powder
a mixing chamber (2) into which the powder particles are thrown and where an aerosol cloud (6a) is formed from incoherent particles,
an inhalation check valve (3), which permits ambient air into the mixing chamber (2) during inhalation to evacuate the particle cloud (6a), and
and exhalation check valve (4), by means of which expired air is led off into the environment to prevent damp breath entering the mixing chamber (2) during exhalation

2. An inhalation device according to claim 1, characterised in that the inhalation and exhalation check valves (3, 4) each comprise a flat, elastic valve element.

3. An inhalation device according to claim 1 or 2, characterised in that the inhalation valve (3) is disposed in the wall of the mixing chamber (2).

4. An inhalation device according to one of claims 1 to 3, characterised in that the exhalation valve (4) is disposed in the wall of a mouthpiece (9).

5. An inhalation device according to one of the preceding claims, characterised in that the oscillator (1) comprises an oscillating membrane (5) on which the powder (6) to be nebulised may be placed.

6. An inhalation device according to one of the preceding claims, characterised in that the oscillator (1) can be activated electrostatically, electromechanically or piezoelectrically.

7. An inhalation device according to one of the preceding claims, characterised in that a sensor (100) for detecting the breath flow rate is provided, said sensor emitting an output signal to a control means (101) which activates the oscillator (1) when a signal value indicating the end of the exhalation phase is achieved.

8. An inhalation device according to one of the preceding claims, characterised in that a display means (102) is provided which is connected with the control means (101) for displaying the evaluation results for the patient.

9. A method for the production of a particulate mist containing a medicament for inhalation by a patient during at least one respiratory cycle comprising the following steps:
detection of the end of the exhalation phase of the respiratory cycle;
placing of a predetermined amount of a powder containing a medicament on the membrane of an oscillator;
activation of the oscillator to deagglomerate the amount of powder during a first stage of the inhalation phase of the respiratory cycle; and
deactivation of the oscillator following completion of the deagglomeration of the amount of powder.

10. A method according to claim 9 having the following further steps:
detection and evaluation of the number of respiratory cycles and inspiratory parameters, and
display of the results of the evaluation.

11. A method according to claim 9 or 10 having the following steps:
registration of the number of breaths and the dosage released, and
display of the applied doses and/or the remaining content in a multi-dose reservoir.
